(19) United States
(12) Patent Application Publication
(10) Pub. No.: US 2021/0128929 A1
(43) Pub. Date: May 6, 2021

(54) Microcatheter Structural Wire with Pacing Function

(71) Applicant: Michael B. McDonald, Cordova, TN (US)

(72) Inventor: Michael B. McDonald, Cordova, TN (US)

(21) Appl. No.: 17/144,638

(22) Filed: Jan. 8, 2021

Related U.S. Application Data
(63) Continuation-in-part of application No. 15/890,767, filed on Feb. 7, 2018.
Application No. 15/890,767 is a continuation-in-part of application No. 15/602,463, filed on May 23, 2017, now Pat. No. 10,898,682.
Application No. 15/602,463 is a continuation-in-part of application No. 15/445,272, filed on Feb. 28, 2017, now Pat. No. 10,898,322.
(60) Provisional application No. 62/958,411, filed on Jan. 8, 2020.
(60) Provisional application No. 62/340,111, filed on May 23, 2016.
(60) Provisional application No. 62/301,270, filed on Feb. 29, 2016.

(51) Int. Cl. A61N 1/375; A61N 1/372

(52) U.S. Cl. CPC A61N 1/37512; A61N 1/37205

(57) ABSTRACT

A microcatheter wire is configured for insertion into a human heart via a human artery. The microcatheter wire has a tube shaft formed from hollow flexible tubing, the tubing comprising an outer plastic layer, a middle conductive layer, and an inner plastic layer. A proximal opening on the tube shaft receives a slideable core. A conductive collar near the proximal end is an area of the tube shaft where the outer plastic layer has been removed and the conductive middle layer is exposed for connection to a pacemaker generator. A distal ring is formed unitarily with the tube shaft, and also has an outer plastic layer, a middle conductive layer, and an inner plastic layer. The distal ring has a plurality of conductive patches along its outer curve where the outer plastic layer has been removed to expose the middle conductive layer for contacting the human heart to allow heart pacing.

### REFERENCE TO RELATED APPLICATIONS

This application claims priority to U.S. Provisional Patent Application No. 62/958,411, entitled “Microcatheter Structural Wire with Pacing Function,” and filed on Jan. 8, 2020. This application is further a continuation-in-part of, and claims priority to, U.S. Non-provisional application Ser. No. 15/890,767, entitled “Hybrid Microcatheter Guidewire, and filed on Feb. 7, 2018, which is a continuation-in-part of, and claims priority to, U.S. Non-provisional application Ser. No. 15/602,463, entitled “Microtube Guide,” and filed on May 23, 2017, which claims priority to Provisional Patent Application U.S. Ser. No. 62/340,111, entitled “Microtube Guide” and filed on May 23, 2016, which is fully incorporated herein by reference. U.S. Non-provisional application Ser. No. 15/602,463 is a continuation-in-part of, and claims priority to, U.S. Non-provisional application Ser. No. 15/445,272, entitled “TAVR Valve Guidewire and Guidetube with Adjustable Distal Loop,” and filed on Feb. 28, 2017, which claims priority to U.S. Provisional Patent Application Ser. No. 62/301,270, entitled “TAVR Valve Guidewire and Guidetube with Adjustable Distal Loop” and filed on Feb. 29, 2016. These patent applications are fully incorporated herein by reference.

### BACKGROUND AND SUMMARY

In the rapidly progressive field of structural heart, there are several different wires available for the delivery of transcatheter valves. The new and unique combination of a microcatheter and movable core wire to function as a structural wire is described in U.S. Publication No. 2018/0161549 A1. The materials and technology of this device make it ideally suited for cardiac pacing as well as device delivery.

During transcatheter valve placement, rapid cardiac pacing is required for the proper and safe positioning of the valve. In most cases of transcatheter valve placement, the cardiac pacing is performed by placing a separate pacing wire in the right ventricle. This pacing approach requires a separate vascular puncture. A bipolar wire that is often balloon tipped for floatation is advanced through the venous system into the right ventricle.

A microcatheter structural wire according to the present disclosure performs the pacing function without requiting a separate vascular puncture. Pacing will be in a unipolar mode, which differentiates it from the present pacing devices used in structural heart. Unipolar cardiac pacing generally results in better stimulation characteristics compared to bipolar pacing. Bipolar pacing has a lower potential for skeletal muscle stimulation. With the unipolar pacing, the distal section of the wire will serve as the cathode and a conductive skin patch will serve as the anode. These cathode and anode circuits are connected to the pacemaker generator.

The present microcatheter structural wire consists of a polyimide microcatheter, a specially engineered moveable core wire, and a flexible distal metal tip. The core wire is designed to interact with the performed distal end of the microcatheter. This dynamic interaction controls the shape of the distal end of the microcatheter structural wire system.

This version of the previously designed microcatheter structural wire can function as a unipolar pacing wire as well as a wire for deployment of the structural heart devices. Designing pacing capacity into the wire will eliminate the use of a separate transvenous pacing system, which adds to the time, risk, and expense of the medical procedure. Having the guidewire function as the cardiac pacing wire will significantly simplify the procedure. Making things simple is usually in the best interest of the patient.

### DESCRIPTION OF THE DRAWINGS

FIG. 1 depicts a microcatheter structural wire system according to an exemplary embodiment of the present disclosure.

FIG. 2 is a cross-sectional view of the microcatheter of FIG. 1, taken along section lines A-A of FIG. 1.

FIG. 3 is a cross-sectional view of the microcatheter of FIG. 1, taken along section lines B-B of FIG. 1.

FIG. 4 is a cross-sectional view of the main shaft of the microcatheter 101 of FIG. 1, taken along section lines C-C of FIG. 1.

FIG. 5 depicts a representation of a patient undergoing a transcatheter valve placement using the microcatheter wire with pacing function as disclosed herein.

### DETAILED DESCRIPTION

FIG. 1 depicts a microcatheter structural wire system 100 according to an exemplary embodiment of the present disclosure. The system 100 comprises a microcatheter structural wire 101 combined with a movable core 102 according to the present disclosure. The microcatheter structural wire 101 is a hollow microcatheter, formed from polyimide plastic embedded with a braided metal in one embodiment.

A proximal opening 107 of the microcatheter structural wire 101 receives the core 102, which slides within the microcatheter structural wire 101 to advance and retract in the direction indicated by directional arrow 120.

The microcatheter structural wire 101 comprises a generally straight main shaft 103 that is hollow to receive the core 102. The microcatheter structural wire 101 further comprises an expandable distal loop 105. The distal loop 105 is disposed at a distal end 106 of the microcatheter structural wire 101. The distal end 106 of the microcatheter structural wire 101 is closed in the illustrated embodiment, and not open like typical microcatheters. The distal end 106 is formed from flexible metal in one embodiment.

The core 102 is designed to interact with the distal end 106 of the microcatheter. This dynamic interaction controls the shape of the distal loop 105 of the microcatheter structural wire, as further discussed herein. The core 102 further comprises a proximal core end 104.

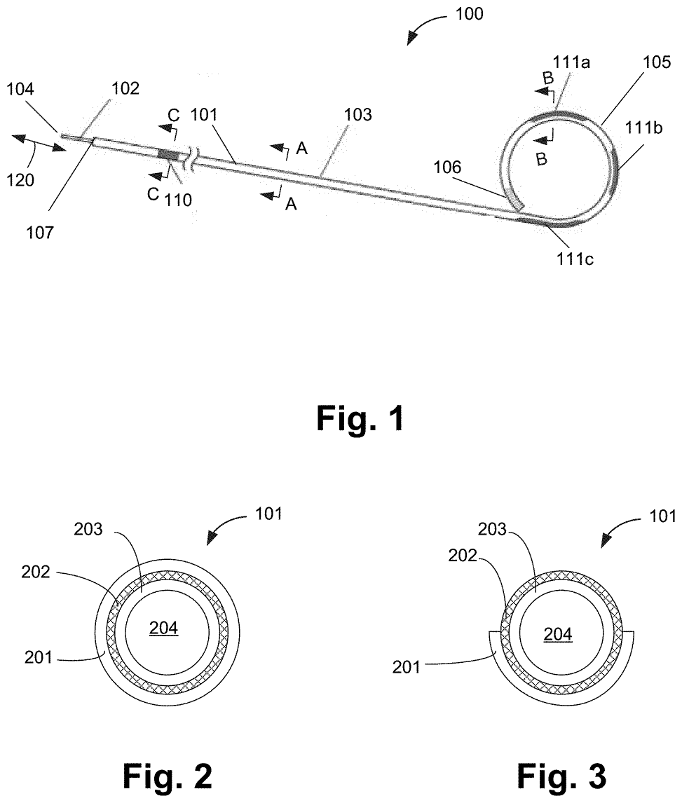

The main shaft 103 of the microcatheter structural wire 101 is formed from kink-resistant, thin-walled, semi-rigid tube that is 0.035 inches in outer diameter and 0.028 inches in inner diameter in one embodiment. The main shaft 103 comprises an inner and outer layer of plastic, and a middle layer of braided metal within the plastic of the guidetube, as further discussed below with respect to FIGS. 2-4. In one embodiment the braided metal layer is steel and tungsten. The plastic layer comprises polyimide in one embodiment. Polyimide has a very low dielectric constant. Most polyimides exhibit dielectric constants in the 2.78-3.48 range and dielectric loss between 0.01 to 0.03 at 1 Hz at room temperature. The braided metal layer is formed from steel and tungsten in one embodiment and tungsten alone in another embodiment. Tungsten is one of the best metals for electrical conductivity. Tungsten shows an electronegativity on the Pauling scale of 2.36. In other embodiments, the braided metal layer may be formed from stainless steel.

In the illustrated embodiment, the body of the distal loop makes about one and one half loops. An outer diameter of the distal loop in this configuration may be about 3.0 centimeters in one embodiment.

When the core 102 is advanced such that its tip (not shown) enters the distal loop 105, the tip contacts an inner surface of the distal loop 105 and causes the diameter of the distal loop 105 to increase. By advancing or retracting the core 102, the size of the distal loop 105 may be enlarged or decreased. Further, the distal loop 105 may fully straighten upon advancement of the core 102 as well. For insertion of the microcatheter 101 into the patient's heart, the core is generally fully advanced to straighten the core. After the microcatheter is in place in the left ventricle, the core is retracted enough that it no longer acts to straighten the distal loop 105, and the distal loop 105 is deployed.

The main shaft 103 comprises a conductive collar 110 where the braided metal layer of the microcatheter is exposed near the proximal end 107 of the microcatheter 101. In this regard, the plastic outer layer is removed in a small section of the main shaft 103 so that the conductive metal braid can be attached to a cathode, as further discussed herein. In one embodiment, the conductive collar is disposed about 10 cm from the proximal end 107 of the microcatheter 101.

The main shaft 103 further comprises a plurality of distal conductive patches 111a, 111b and 111c where the braided metal layer is also exposed. In the illustrated embodiment, the distal conductive patches 111a, 111b and 111c are rectangular areas extending partially around a circumference of the distal end 105 where the plastic outer layer is removed. The purpose of the distal conductive patches is to contact the inside of the patient's left ventricle and provide a pacing function, as further discussed herein. The distal conductive patches 111a, 111b, and 111c are located on the outer curvature of the distal loop 105.

Each of the distal conductive patches 111a, 111b and 111c is a rectangular area about 2-3 cm long in one embodiment. The illustrated embodiment has three distal conductive patches 111a, 111b, and 111c as shown, to provide good conductivity to the patient's heart. Other sizes and numbers of conductive patches are possible, provided that they provide sufficient conductivity.

In operation of the unipolar pacing system according to the present disclosure, the cathode will be the exposed braided metal layer at the distal conductive patches 111a, 111b, and 111c. The electrical current connection will be at the exposed braid of the conductive collar 110. In this regard, a wire and clip (not shown) may be attached to the conductive collar 110, and this connection will be the negative polarity (cathode) of the unipolar pacing system.

The anode (positive) part of the system will consist of a wire attached to a conductive skin patch, as further discussed herein with respect to FIG. 5.

FIG. 2 is a cross-sectional view of the microcatheter 101 of FIG. 1, taken along section lines A-A of FIG. 1. This view does not include the core 102 (FIG. 1) within the microcatheter 101. The microcatheter 101 comprises a plastic outer layer 201 and a plastic inner layer 203 with a braided metal layer 202 embedded within the plastic layers 201 and 203. The inside 204 of the microcatheter 101 is hollow to receive the core 102 (FIG. 1).

FIG. 3 is a cross-sectional view of the microcatheter 101 of FIG. 1, taken along section lines B-B of FIG. 1, specifically at the location of the distal conductive patch 111a (FIG. 1). As shown in this view, the plastic outer layer 201 has been removed from the microcatheter 101 over about half of the circumference of the microcatheter 101 (i.e., about 180 degrees around the microcatheter). In other embodiments, more or less of the plastic outer layer may be removed, provided that sufficient area of the braided metal layer 202 is exposed to provide good conductivity to the patient's heart.

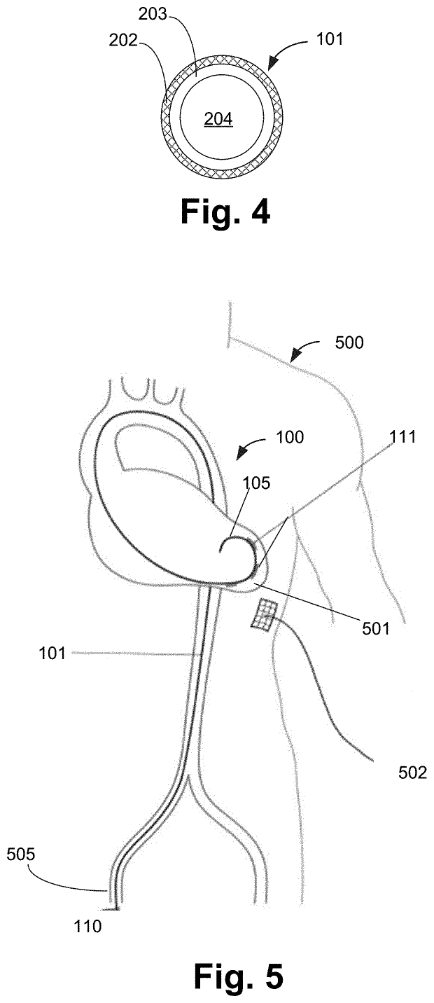

FIG. 4 is a cross-sectional view of the main shaft 103 of the microcatheter 101 of FIG. 1, taken along section lines C-C of FIG. 1, specifically at the location of the conductive collar 110 (FIG. 1). At the location of conductive collar 110, the entire plastic outer layer 201 (FIGS. 2-3) has been removed around the entire circumference of the microcatheter 101, leaving the braided metal layer 202 exposed.

FIG. 5 depicts a representation of a patient 500 undergoing a transcatheter valve placement using the microcatheter wire with pacing function system 100 as disclosed herein. The microcatheter structural wire 101 has been threaded through the patient's femoral artery 505 into the patient's left ventricular apex 501. Vessels other than the femoral artery may be used if needed. The microcatheter structural wire 101 serves as the guide for delivery of a valve deployment device with an attached valve (not shown). Once the microcatheter structural wire 101 has been placed in the left ventricle, and the delivery device with the transcatheter valve is properly positioned across the native valve, the distal ring 105 is deployed as discussed herein and rapid ventricular pacing is initiated. The rapid pacing aids in the exact positioning of the new valve (not shown) and its safe deployment. As soon as the valve deployment is completed, the pacing is terminated.

As discussed above, the cathode for the pacing is the exposed braided metal layer at the distal conductive patches 111 in the left ventricle of the patient. The electrical current connection will be at the exposed braid of the conductive collar 110. The anode (positive) part of the system will consist of a wire (not shown) attached to a conductive skin patch 502. This patch 502 will be applied to the skin on the left side of the chest not far from the left ventricular apex. The electron flow will be from the cathode to the anode.

The electric wires from the cathode and anode will be connected to a pacemaker generator (not shown). When the necessary pacing is completed, the cathode wire/clip is easily removed from the proximal end of the microcatheter.

An alternative design is to use metal conductive collars on the distal loop of the microcatheter in place of just having exposed braid. These collars will be on the outer layer of the microcatheter and electrically connected to the braided metal layer. The collars will provide strength to the microcatheter and an improved surface contact area. A similar collar will be on the proximal end of the microcatheter for the connection with the pulse generator wire.

This disclosure may be provided in other specific forms and embodiments without departing from the essential characteristics as described herein. The embodiments described are to be considered in all aspects as illustrative only and not restrictive in any manner.

What is claimed is:

1. A microcatheter wire configured for insertion into a human heart via a human artery, the microcatheter wire comprising:
a tube shaft formed from substantially hollow flexible tubing, the tubing comprising an outer plastic layer, a middle conductive layer, and an inner plastic layer, the tube shaft comprising a proximal opening on a proximal end of the tube shaft, the tube shaft further comprising a conductive collar spaced apart from and near the proximal end, the conductive collar comprising an area of the tubing where the outer plastic layer is removed and the conductive middle layer is exposed, the conductive collar configured to connect to a pacemaker generator;
a distal ring formed unitarily with and extending from the tube shaft, the distal ring formed from substantially hollow flexible tubing, the tubing comprising an outer plastic layer, a middle conductive layer, and an inner plastic layer, the distal ring comprising a plurality of conductive patches along an outer curve of the distal ring where the outer plastic layer has been removed to expose the middle conductive layer for contacting the human heart.

2. The microcatheter wire of claim 1, wherein the outer plastic layer and the inner plastic layer of the tube shaft and distal ring are formed from polyimide.

3. The microcatheter wire of claim 1, wherein the middle conductive layer of the tube shaft and distal ring are formed from braided metal.

4. The microcatheter wire of claim 3, wherein the braided metal is formed from tungsten.

5. The microcatheter wire of claim 1, wherein the plurality of conductive patches comprises three rectangular patches, the conductive patches spaced apart from one another by between 0.5 and 1 centimeters.

6. The microcatheter wire of claim 1, wherein each of the plurality of conductive patches is formed by lasering away the outer layer of tubing partially around a circumference of the tubing.

7. The microcatheter wire of claim 1, the conductive collar spaced apart from the proximal end by between 5 and 10 centimeters.

8. The microcatheter wire of claim 1, wherein the distal ring is in a same plane as the tube shaft.

9. The microcatheter wire of claim 1, further comprising a flexible core receivable by the proximal opening of the tube shaft of the microcatheter wire and slideable within the microcatheter wire, the microcatheter wire and the core configured such that partially advancing the core within the microtube adjusts a shape of the distal ring, and fully advancing the core within the microtube substantially straightens the microtube.

10. The device of claim 9, the core configured to cause the distal ring of the microtube to deploy when the core is retracted from the distal ring, the core further configured to cause the diameter of the distal ring to increase when the core is partially advanced into the distal ring.

11. A microcatheter wire configured for insertion into a human heart via a human artery, the microcatheter wire comprising:
a tube shaft and a distal ring, the tube shaft configured to conduct electricity from the plurality of conductive patches to a pacemaker generator, the tube shaft and distal ring formed from substantially hollow flexible tubing, the tubing comprising an outer plastic layer, a middle conductive layer, and an inner plastic layer, the distal ring extending from the tube shaft in a same plane as the tube shaft, the distal ring comprising a plurality of conductive patches along an outer curve of the distal ring, the conductive patches electrically connected to the middle conductive layer.

12. The microcatheter of claim 11, where the conductive patches comprise areas on the distal ring where the outer plastic layer has been removed to expose the middle conductive layer for contacting the human heart.

13. The microcatheter wire of claim 11, the tube shaft comprising a proximal opening on a proximal end of the tube shaft, the tube shaft further comprising a conductive collar spaced apart from and near the proximal end, the conductive collar comprising an area of the tubing where the outer plastic layer is removed and the conductive middle layer is exposed, the conductive collar configured to connect to a pacemaker generator.

14. The microcatheter wire of claim 11, wherein the outer plastic layer and the inner plastic layer of the tube shaft and distal ring are formed from polyimide.

15. The microcatheter wire of claim 11, wherein the middle conductive layer of the tube shaft and distal ring are formed from braided metal.

16. The microcatheter wire of claim 15, wherein the braided metal is formed from tungsten.

17. The microcatheter wire of claim 11, wherein the plurality of conductive patches comprises three rectangular patches, the conductive patches spaced apart from one another by between 0.5 and 1 centimeters.

18. The microcatheter wire of claim 11, wherein each of the plurality of conductive patches is formed by lasering away the outer layer of tubing partially around a circumference of the tubing.

19. The microcatheter wire of claim 11, further comprising a flexible core receivable by the proximal opening of the tube shaft of the microcatheter wire and slideable within the microcatheter wire, the microcatheter wire and the core configured such that partially advancing the core within the microtube adjusts a shape of the distal ring, and fully advancing the core within the microtube substantially straightens the microtube.

20. The device of claim 19, the core configured to cause the distal ring of the microtube to deploy when the core is retracted from the distal ring, the core further configured to cause the diameter of the distal ring to increase when the core is partially advanced into the distal ring.
